(19) United States
(12) Patent Application Publication
(10) Pub. No.: US 2008/0172038 A1
(43) Pub. Date: Jul. 17, 2008

(54) STEERABLE CATHETER SYSTEM

(76) Inventors: Mike Dollar, Garland, TX (US); Victor Dubuclet, Dallas, TX (US)

Correspondence Address: CARSTENS & CAHOON, LLP, P O BOX 802334, DALLAS, TX, 75380, US

(21) Appl. No.: 11/550,485

(22) Filed: Oct. 18, 2006

(51) Int. Cl. A61M 25/00

(52) U.S. Cl. 604/528

(57) ABSTRACT

The present invention is directed towards a steerable multi-lumen catheter system with a locking mechanism and a plurality of ports connected to the lumens inside the multi-lumen catheter.

### FIELD OF THE INVENTION

This invention relates to steerable catheters and methods for using such catheters for treating paranasal sinuses.

### BACKGROUND OF THE INVENTION

In order to fully understand this invention, it is important to consider the anatomy of the sinus system. The sinus system consists of many different pathways, called ducts or ostia, which allow mucus, air and other substances to drain and flow through the system. Inflammation can occur in the tissues that make up the ducts and ostia, causing them to swell and block the normal flow. Inflammation may be caused by allergies, noxious agents, nasal polyps, and other factors. Over time there can be a pathologic increase in inflamed tissue causing permanent disruption in the flow through the sinus system. Obstruction of the narrow ducts and ostia between the paranasal sinuses and nasal cavity develops, resulting in a vicious cycle of increased secretions, edema and ultimately complete blockage of the sinus pathways. The state of chronic sinus inflammation is called sinusitis.

Treatment with antibiotics, corticosteroids in nasal sprays or systematically, and antihistamines may result in effective resolution of sinusitis. However, some patients become resistant to medical treatment and surgery becomes necessary. Endoscopic sinus surgery is performed from an intranasal approach, thus eliminating the need for external incisions. A minimally invasive type of endoscopic surgery, called balloon catheterization or sinuplasty, involves placing an expandable device, such as a deflated balloon, inside the clogged sinus pathways and inflating the balloon in order to open the clogged pathway. This type of surgery has also been used to open clogged pathways in other body systems, including in the vascular system, the urinary tract, and the lacrimal system. Some catheters are “steerable catheters” in that they incorporate a means that allows surgeons to deflect the tip in at least one direction, thereby allowing the surgeon to “steer” the tip of the catheter to the region of interest inside the body. Steerable catheters typically contain one or two small lumens that run the length of the catheter and house steering wires. The steering wires are attached to the tip of the catheter so that pulling on one of the steering wires will deflect the tip in the direction of the steering wire.

### SUMMARY OF THE INVENTION

The present invention is thus directed towards an improved steerable catheter. The steerable catheter of the present invention is ergonomically shaped to fit a human hand to make endoscopic surgery easier. It is shaped like a pistol, with a pistol grip, a steering mechanism located where the trigger of a hand gun is located, and a catheter corresponding to the barrel of a hand gun. The steerable catheter of the present invention incorporates several features that are improvements over the prior art.

First, the steerable catheter uses a novel rocker switch steering trigger, or “rocker steering disc”, with teeth built into one of its surfaces. The rocker steering disc is shaped like a cylinder with a wedge shaped piece removed. It is mounted on the grip of the steerable catheter so that the surgeon can rotate it in one direction or the other using one or two fingers. It also incorporates teeth into its curved surface that interact with the locking mechanism.

The locking mechanism is a spur attached to an arm, mounted on the handle. The arm rotates at a point opposite the spur, and has two positions: lock and unlock. In the locked position, the spur engages the teeth of the rocker steering disc, holding the rocker steering disc in position. In the unlocked position, the spur disengages the teeth and allows the rocker steering disc to rotate freely. The locking mechanism protrudes from the steerable catheter handle on both sides of the handle, allowing it to be operated by either the right or left thumb, depending on which hand is holding the steerable catheter.

The present invention also incorporates three or four ports of entry into a multi-lumen catheter. The location of the ports of entry can vary depending on the use each will be subject to. One embodiment has all of the ports located above the handle. Another embodiment places at least one port at the bottom of the handle. Locating ports at the top of the handle allows a surgeon to easily switch instruments traveling through each lumen of the multi-lumen catheter (such as a balloon catheter), while ports located at the bottom of the handle are best for devices that will be used throughout the entire surgery and not switched out by the surgeon (such as a vacuum).

### BRIEF DESCRIPTION OF THE DRAWINGS

A more complete understanding of the method of the present invention may be had by reference to the following detailed description when taken in conjunction with the accompanying drawings, wherein:

FIG. 1A is an angled view of the present invention;

FIG. 1B is a different angled view of the present invention;

FIG. 2A is a profile view of the present invention with all ports located above the handle;

FIG. 2B is a profile view of the present invention with one port located at the bottom of the handle;

FIG. 2C is a profile view of the present invention with two ports located at the bottom of the handle;

FIG. 3A is an angled view of the present invention depicting four ports of entry located above the handle;

FIG. 3B is an angled view of the present invention depicting three ports of entry located above the handle;

FIG. 4A is a cross-sectional view of the present invention with ports of entry located above the handle;

FIG. 4B is a cross-sectional view of the present invention with one port of entry located at the bottom of the handle;

FIG. 4C is a cross-sectional view of the present invention with two ports of entry located at the bottom of the handle;

Where used in the various figures of the drawing, the same numerals designate the same or similar parts. Furthermore, when the terms “top,” “bottom,” “first,” “second,” “upper,” “lower,” “height,” “width,” “length,” “end,” “side,” “horizontal,” “vertical,” and similar terms are used herein, it should be understood that these terms have reference only to the structure shown in the drawing and are utilized only to facilitate describing the invention.

All figures are drawn for ease of explanation of the basic teachings of the present invention only; the extensions of the figures with respect to number, position, relationship, and dimensions of the parts to form the preferred embodiment will be explained or will be within the skill of the art after the following teachings of the present invention have been read and understood. Further, the exact dimensions and dimensional proportions to conform to specific force, weight, strength, and similar requirements will likewise be within the skill of the art after the following teachings of the present invention have been read and understood.

### DETAILED DESCRIPTION OF THE INVENTION

The present invention is directed towards a steerable catheter system used for endoscopic surgery.

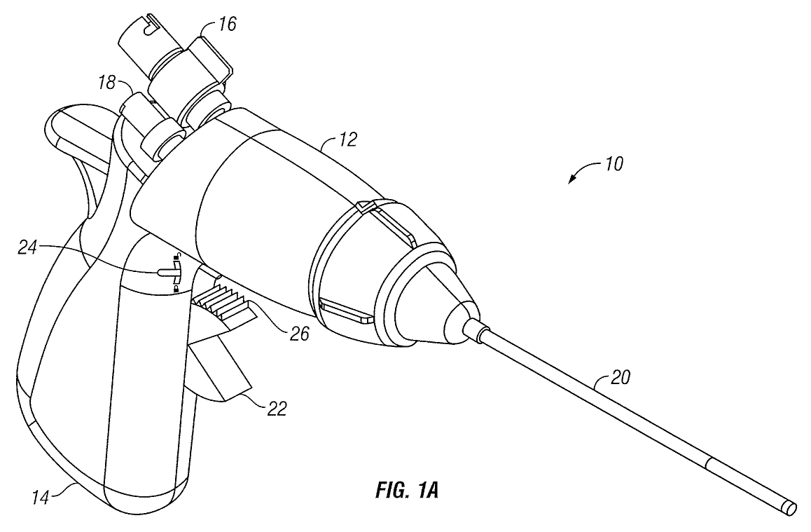

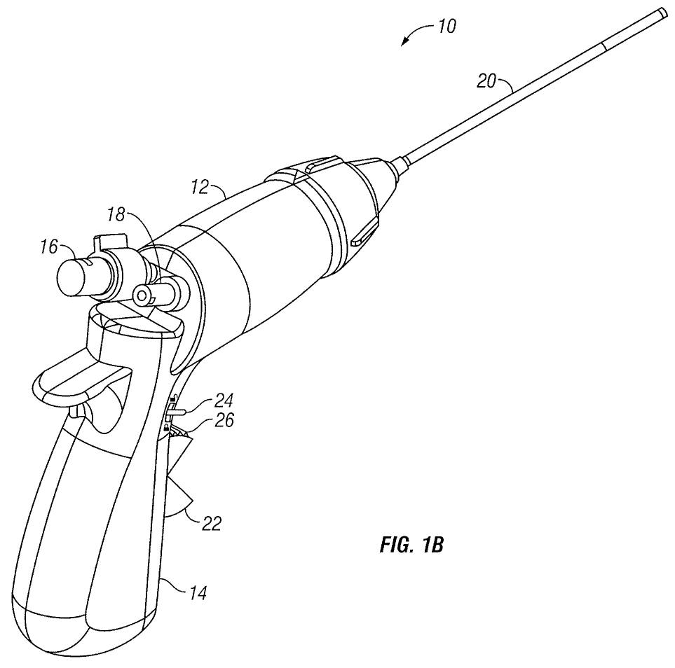

Referring initially to FIG. 1A, therein is depicted the steerable catheter system 10 of the present invention. The steerable catheter system 10 is comprised of an ergonomically shaped housing unit 12 with a pistol grip handle 14, at least two ports of entry 16 and 18 connected to a multi-lumen steerable catheter 20, a rocker steering mechanism 22, and a locking mechanism 24. The rocker steering mechanism 22 is a semi-cylindrical steering disc (shaped like a cylinder missing a wedge-shaped portion) and is rotably mounted on the handle 14 with the wedge-shaped portion facing away from the handle 14. The rocker steering mechanism 22 also incorporates a series of teeth 26 into its curved surface. The lines defining the apexes of the teeth 26 are approximately radially equidistant from and approximately parallel to the axis of rotation of the rocker steering mechanism 22 and are approximately evenly spaced along the curved surface of the rocker steering disc 22. The locking mechanism 24 can be engaged and disengaged from either side of the handle 14 because openings on each side of the handle provide allow either the right thumb or left thumb to engage or disengage the locking mechanism 24. FIG. 1A also depicts two ports of entry 16 and 18 above the handle 14. During endoscopic surgery, a surgeon inserts a medical device into one of the ports, through the tube connecting the port to the multi-lumen steerable catheter 20, and

Referring now to FIG. 1B, therein is depicted a rear angled view of the embodiment of present invention depicted in FIG. 1A. Two ports of entry 16 and 18, the locking mechanism 24, and the rocker steering mechanism 22 with teeth 26 are depicted.

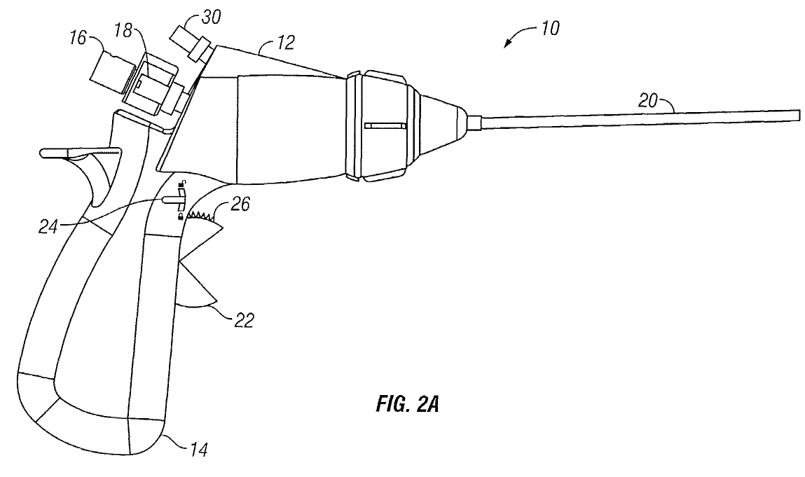

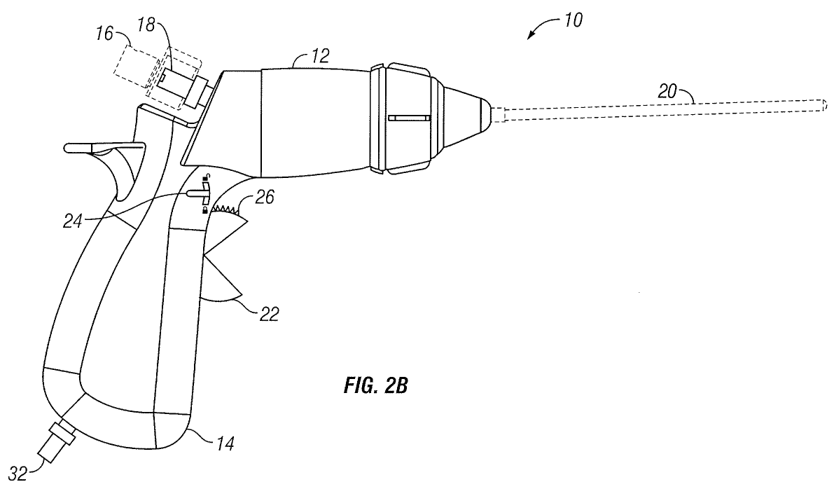

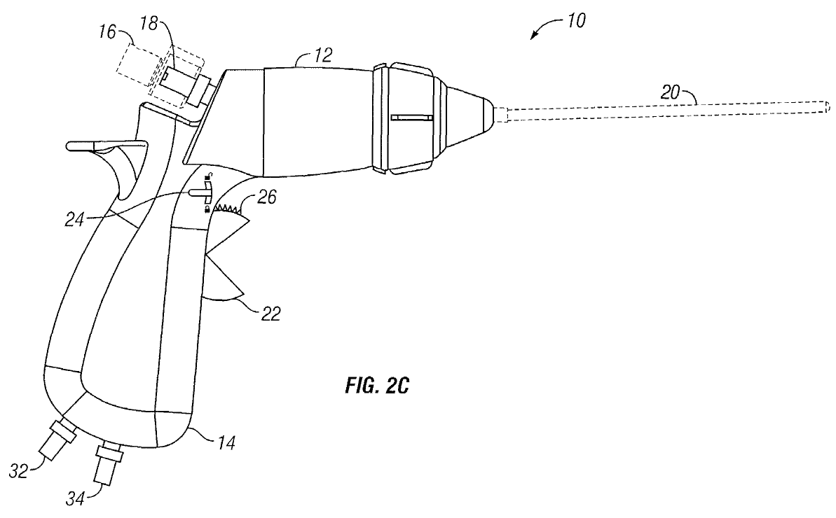

Referring next to FIG. 2A, therein is depicted a profile view of a second embodiment of the present invention. In this embodiment, three ports 16, 18, and 30 are provided above the handle 14. The locking mechanism 24, rocker steering disc 22 with teeth 26 are also depicted.

Referring next to FIG. 2B, therein is depicted another embodiment of the present invention. Here, three ports 16, 18 and 32 are depicted: two ports 16 and 18 above the handle 14 and one port 32 at the bottom of the handle 14. Having a port 32 located at the bottom of the handle 14 is more convenient for some surgical devices that are used throughout the surgical procedure, for example a vacuum. The bottom location is more useful than a top location for ports housing such surgical devices because it keeps the more conveniently located top ports of entry less cluttered and thus more accessible to the surgeon. The surgeon is able to insert those medical devices that will be used throughout the surgery into the bottom ports of entry leaving the top ports of entry open and accessable.

Referring next to FIG. 2C, therein is depicted another embodiment of the present invention. Here, four ports 16, 18, 32, 34 are depicted: two ports 16 and 18 above the handle 14 and two ports 32 and 34 at the bottom of the handle 14. The ports at the bottom of the handle in this embodiment give the same advantages to a surgeon as the embodiment depicted in FIG. 2B, with the added functionality of another port.

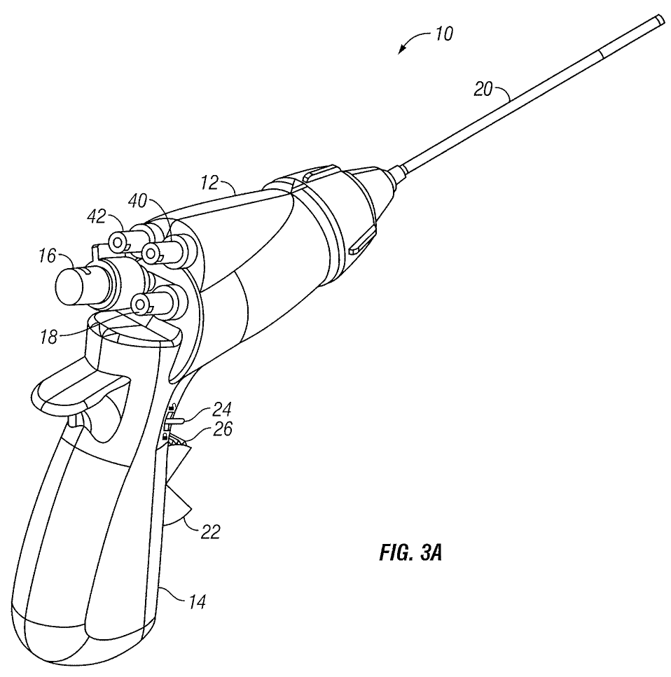

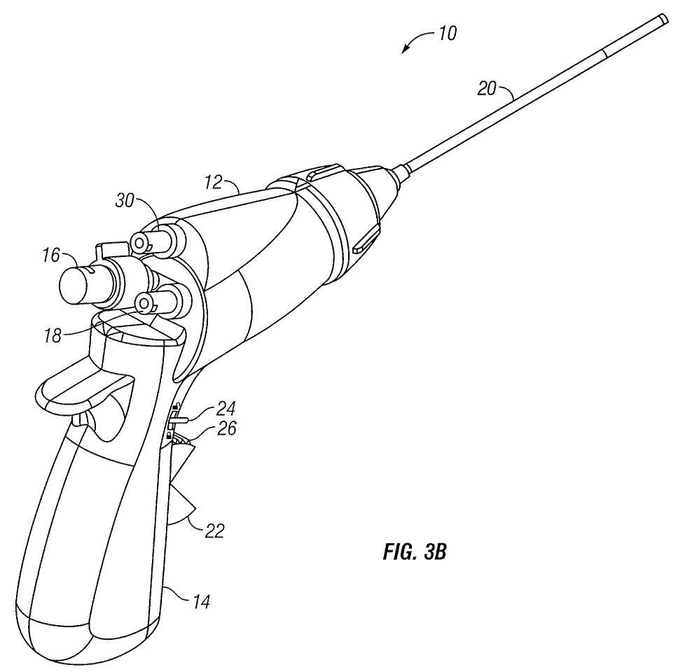

Referring to FIGS. 3A and 3B, therein are depicted the four-port and three-port embodiments of the present invention, respectively. In these embodiments, all of the ports 16, 18, 40, and 42 are located above the handle 14. These embodiments are useful for surgeries involving several different medical instruments that will be interchanged frequently during surgery because all of the ports are easily accessible to the surgeon above the handle 14.

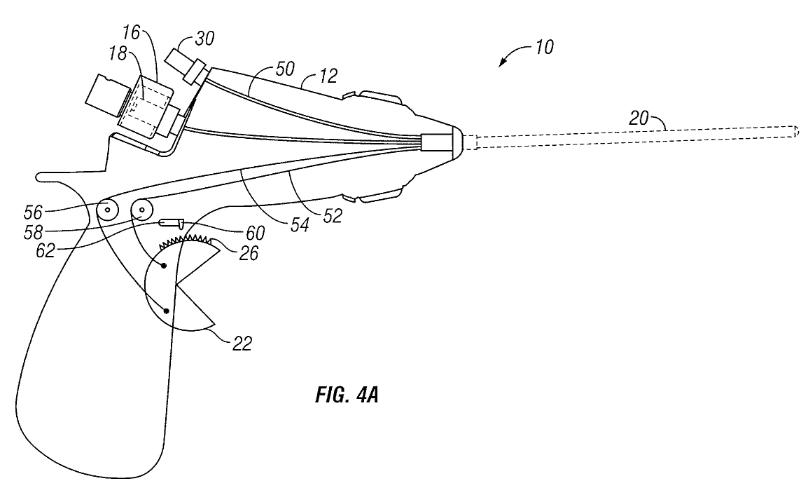

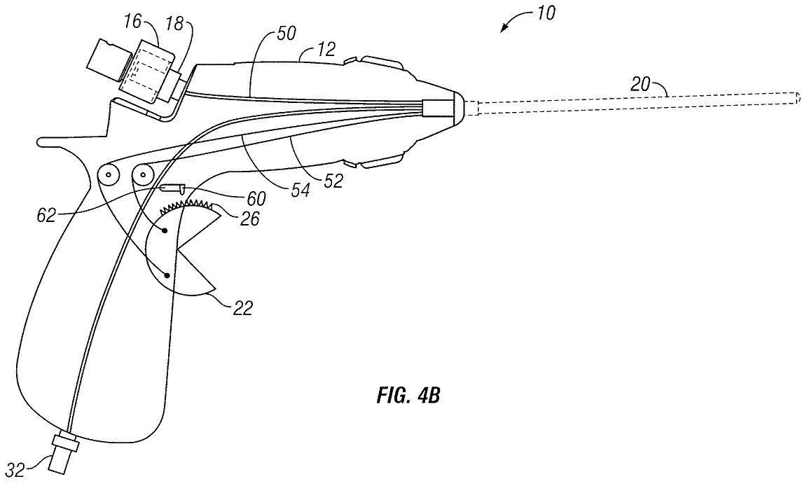

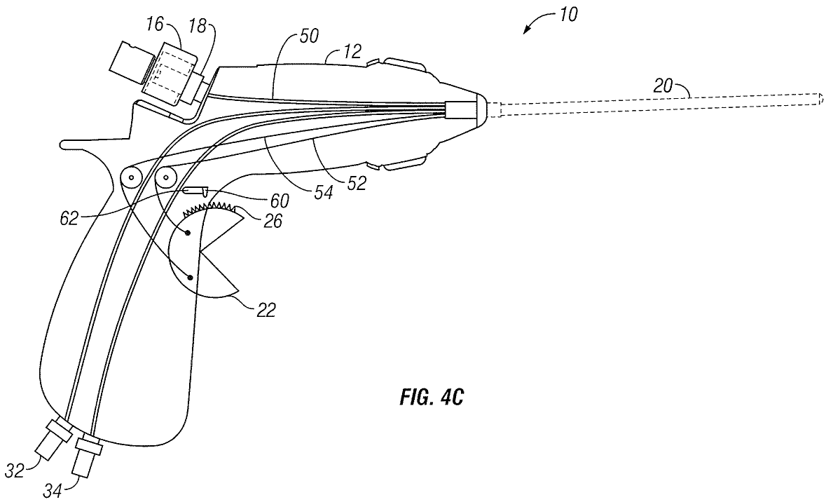

Referring next to FIG. 4A, therein is depicted a cross section of the present invention showing the interior of the housing unit 12 and handle. A flexible tube 50 connects each port of entry 16, 18 and 30 to a corresponding lumen inside the multi-lumen catheter 20. The multi-lumen catheter 20 also contains two small lumens that house two steering wires 52 and 54 attached to the tip of the catheter. Each steering wire 52 and 54 runs the length of the catheter 20, engages a pulley 56 and 58, and attaches to the rocker steering disc 22. The first steering 54 wire attaches to the rocker steering disc 22 below the axis of rotation for the rocker steering disc (between the axis of rotation for the rocker steering disc and the bottom of the handle). The second steering wire 52 attaches to the rocker steering disc 22 at a point above the axis of rotation for the rocker steering disc. If the rocker steering disc 22 is rotated in one direction, the first steering wire 54 is put under tension while the second steering wire 52 is put under compression, resulting in the tip of the multi-lumen catheter 20 being deflected in the direction of the first steering wire 54. If the rocker steering disc 22 is rotated in the opposite direction, similar principles will deflect the tip in the direction of the second steering wire 52. A spur 60 attached to an arm 62 is provided as a part of the locking mechanism. The arm 62 is rotably mounted on the handle near the rocker steering disc 22. The spur 60 engages the teeth 26 when the arm 62 is rotated in the direction of the rocker steering disc 22 (the “lock” position), and disengages the teeth 26 when the arm 62 is rotated away from the rocker steering disc 22 (the “unlock” position). At any time during the rotation of the rocker steering disc 22, the arm 62 and spur 60 of the locking mechanism can be rotated towards the rocker steering disc 22 and engage the teeth 26, locking the rocker steering disc 22 and the catheter tip in place. The arm 62 and spur 60 can be disengaged at any time by rotating them away from the rocker steering disc 22.

Referring next to FIGS. 4B and 4C, therein are depicted two different cross-sections of different embodiments of the present invention. The only difference between these embodiments and the embodiment depicted in FIG. 4A is that the port(s) of entry 32 and 34 are located at the bottom of the handle. The functionality of the different parts of the steerable catheter system 10 remain unchanged.

We claim:

1. A steerable catheter system comprising:
(a) a housing unit adapted to be hand-held and having a top surface, a back end, and a bottom surface;
(b) a multi-lumen catheter having a proximal end connected to said housing unit, an opposite distal end and a plurality of lumens extending longitudinally therethrough;
(c) a plurality of ports on said housing unit;
(d) one flexible tube connecting each said port to an individual said lumen at the proximal end of said multi-lumen catheter;
(e) a steering means for manipulating the distal end of said multi-lumen catheter, said steering means comprising
(i) a semi-cylindrical steering disc mounted inside said housing unit and extending outside said housing unit;
(ii) two steering wires each with a distal end and a proximal end, the proximal ends being separately attached to said steering disc and the distal ends each being separately attached to the distal end of said multi-lumen catheter at predetermined opposing points;
(f) a locking means on said housing unit;

2. The steerable catheter of claim 1 wherein said locking means comprises:
(i) a multitude of teeth along a curved surface of said steering disc.
(ii) a spur attached to an arm mounted inside said housing unit such that movement of said arm towards said steering disc causes said spur to engage said teeth and that movement of said arm away from said steering disc causes said spur to disengage said teeth, said spur being accessible from outside said housing unit.

3. The steerable catheter of claim 1 wherein said ports are located on said back end of said housing unit.

4. The steerable catheter of claim 1 wherein two said ports are located on said back end of said housing unit and two said ports are located on said bottom surface of said housing unit.

5. The steerable catheter of claim 1 wherein three said ports are located on said back end of said housing unit and one said port is located on said bottom surface of said housing unit.
